(19) United States
(12) Patent Application Publication
(10) Pub. No.: US 2004/0127746 A1
(43) Pub. Date: Jul. 1, 2004

(54) Catalyst for synthesizing unsaturated aldehyde and unsaturated carboxylic acid, method of preparing same, and method of synthesizing unsaturated aldehyde and unsaturated carboxylic acid with the catalyst

(76) Inventors: Masahide Kondo, Hiroshima (JP); Seiichi Kawato, Hiroshima (JP); Toru Kuroda, Hiroshima (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/473,255

(22) PCT Filed: Mar. 27, 2002
(86) PCT No.: PCT/JP02/02941

(30) Foreign Application Priority Data
Mar. 27, 2001 (JP) 2001-090321
Mar. 30, 2001 (JP) 2001-100319

(51) Int. Cl. C07C 51/16; C07C 45/28

(52) U.S. Cl. 562/546; 502/311; 568/479

(57) ABSTRACT

The present invention provides novel extrusion-molded catalysts which is suitably usable in the synthesis of an unsaturated aldehyde and an unsaturated carboxylic acid by vapor-phase catalytic oxidation, which are prepared by molding-molding catalyst particles (or powders) comprising at least molybdenum, bismuth and iron as metallic elements participating in its catalytic action on this reaction, and which exhibit high catalytic activity and high selectivity for the unsaturated aldehyde and unsaturated carboxylic acid being desired products. When previously formed catalyst particles are subjected to extrusion molding, the catalyst particles are kneaded, for example, with a β-1,3-glucan and a liquid. Then, the kneaded material is subjected to an extrusion molding step in which a ceramic material is used for at least a part of the catalyst flow path. Thereafter, the extrusion-molded material is dried, calcined and otherwise treated to obtain a final extrusion-molded catalyst.

### TECHNICAL FIELD

This invention relates to an extruded catalyst for catalyzing a vapor-phase catalytic oxidation reaction in which an unsaturated aldehyde and an unsaturated carboxylic acid are synthesized through the vapor-phase catalytic oxidation by using propylene, isobutylene, tert-butyl alcohol (hereinafter referred to TBA) or methyl tert-butyl ether (hereinafter referred to MTBE) as a raw material with molecular oxygen as an oxygen source. More particularly, it relates to a catalyst for synthesizing an unsaturated aldehyde and an unsaturated carboxylic acid, which is prepared by extrusion-molding catalyst particles (or powders) containing at least molybdenum, bismuth and iron as metallic elements participating in its catalytic action on the aforesaid vapor-phase catalytic oxidation reaction, a process for preparing such catalysts by extrusion, and a process for the synthesis of an unsaturated aldehyde and an unsaturated carboxylic acid by utilizing such an extruded catalyst.

### BACKGROUND ART

Conventionally, a large number of propositions have been made with respect to a solid catalyst usable for the process where an unsaturated aldehyde and an unsaturated carboxylic acid is synthesized through the vapor-phase catalytic oxidation by using propylene, isobutylene, TBA or MTBE as a raw material with molecular oxygen as an oxygen source, and a process for preparing such a solid catalyst.

Many of these catalysts for vapor-phase catalytic oxidation reactions, which are usable for the production of unsaturated aldehydes and unsaturated carboxylic acids, have components containing at least molybdenum, bismuth and iron as metallic elements participating in their catalytic action. Moreover, for industrial purposes, there are used shaped catalysts that are formed out of catalyst particles (or powders) having the aforesaid components by a desired shape-pattern. According to the shaping method, these shaped catalysts are classified into extrusion-molded catalyst, carrier-supported catalyst and so on. Usually, the extrusion-molded catalyst is prepared by a process comprising the steps of kneading previously formed particles comprising catalyst components together with a liquid medium and extrusion-molding this kneaded material. On the other hand, the carrier-supported catalyst is prepared by a process comprising the step of supporting previously prepared powders containing catalyst components on a carrier.

With respect to extrusion-molded catalysts, various techniques for improving their catalytic properties have been proposed; which include, for example, a process for the extrusion in which graphite or an inorganic fiber is added to the chief ingredient comprising particles containing catalyst components, for example, during kneading, and thereby a molded catalyst obtained has a improved mechanical strength or selectivity for the product of the reaction catalyzed by it (Japanese Patent Laid-Open No. 150834/'85); a molded product having a specified shape and specified properties (Japanese Patent Publication No. 36740/'87); and a process for the extrusion in which a certain cellulose derivative is added to the kneaded material prior to the extrusion step of a catalyst (Japanese Patent Laid-Open No. 16464/'95).

However, many of these published propositions are concerned with the choice of an additive added to the kneaded material during extrusion molding process, and few of them mention the details of the process for preparing a catalyst by extrusion-molding a kneaded material. While some of the extruded catalysts prepared by these known methods are being applied to actual industrial production, desired are such further improvements in catalytic activity, selectivity for target products from the viewpoint of industrial catalysts on aiming at more efficient production.

### DISCLOSURE OF THE INVENTION

Accordingly, the present invention will solve the objects described above, and thus it is the aim of the invention to provide a novel catalyst for the synthesis of an unsaturated aldehyde and an unsaturated carboxylic acid which is a molded catalyst catalyzing a reaction where an unsaturated aldehyde and an unsaturated carboxylic acid is synthesized by vapor-phase catalytic oxidation reaction from a raw material having a corresponding carbon chain by using molecular oxygen as an oxygen source and which exhibits high catalytic activity as well as high selectivity for the unsaturated aldehyde and unsaturated carboxylic acid being aimed products; a process for preparing said catalyst conveniently; and a process adapted for synthesizing a targeted unsaturated aldehyde and unsaturated carboxylic acid with high selectivity by using this catalyst. More specifically, an aim of the present invention is to provide a novel catalyst for the synthesis of an unsaturated aldehyde and an unsaturated carboxylic acid which is suitably applicable to the process wherein by using propylene, isobutylene, TBA or MTBE as a raw material with molecular oxygen as an oxygen source, an unsaturated aldehyde and an unsaturated carboxylic acid having a corresponding carbon chain is synthesized through vapor-phase catalytic oxidation and which is prepared by extrusion-molding catalyst particles (or powders) containing at least molybdenum, bismuth and iron as metallic elements participating in its catalytic action on said vapor-phase catalytic oxidation reaction, and which exhibits high catalytic activity and superior selectivity for the unsaturated aldehyde and unsaturated carboxylic acid being aimed products; a process for preparing said catalyst conveniently; and a process adapted for synthesizing a targeted unsaturated aldehyde and unsaturated carboxylic acid with high selectivity by using said catalyst.

The present inventors carried out intensive research and investigations with a view to solving the problems described above, and have now found that, with respect to an extrusion-molded catalyst usable for the process where an unsaturated aldehyde and an unsaturated carboxylic acid is synthesized by a vapor-phase catalytic oxidation reaction using molecular oxygen as an oxygen source, when the extrusion molding step itself is especially modified in addition to the choice of a composition for the catalyst particles composing it and of an additive added to a kneaded material containing the catalyst particles to be extrusion-molded, an extrusion-molded catalyst prepared from the same catalyst particles can achieve an improvement in catalytic activity and in selectivity for the unsaturated aldehyde and unsaturated carboxylic acid being aimed products. More specifically, the present inventors have found that, when a small amount of a β-1,3-glucan is added to a kneaded material containing previously prepared catalyst particles and being subjected to extrusion molding, the resulting extrusion-molded catalyst exhibits higher catalytic activity and higher selectivity for the unsaturated aldehyde and unsaturated carboxylic acid being aimed products, and that, when a ceramic material is utilized for at least a part of the path for catalyst flow in the extrusion molding step, the resulting extrusion-molded catalyst exhibits higher catalytic activity and higher selectivity for the unsaturated aldehyde and unsaturated carboxylic acid being desired products, as compared with the conventional case where a catalyst flow path made of metal is used. The present invention has been completed on the basis of these findings.

That is, a catalyst for the synthesis of an unsaturated aldehyde and an unsaturated carboxylic acid in accordance with a first embodiment of the present invention is characterized in that said catalyst is an extrusion-molded catalyst comprising at least molybdenum, bismuth and iron as metallic elements participating in its catalytic action on the vapor-phase catalytic oxidation reaction in which it is usable to catalyzes the vapor-phase catalytic oxidation reaction for the synthesis of the unsaturated aldehyde and the unsaturated carboxylic acid by using propylene, isobutylene, tert-butyl alcohol or methyl tert-butyl ether as a raw material and using molecular oxygen as an oxygen source, and said extrusion-molded catalyst being extruded in the step where, when previously prepared catalyst particles containing at least molybdenum, bismuth and iron are subjected to extrusion molding, a ceramic material is used for at least a part of the catalyst flow path in said step of extrusion-molding a kneaded material containing said catalyst particles. In this embodiment, it is preferable that said kneaded material be obtained by adding a β-1,3-glucan and a liquid to the catalyst particles and kneading the resulting mixture. Further, it is more preferable that said kneaded material be obtained by adding a β-1,3-glucan, a cellulose derivative and a liquid to the catalyst particles and kneading the resulting mixture. On the other hand, in the first embodiment of the present invention, it is preferable that the ceramic material used for at least a part of the catalyst flow path in the extrusion molding step comprise a ceramic material selected from the group consisting of zirconia, alumina, silica, titania and mixtures of two or more of these materials.

In the first embodiment of the present invention, the process for preparing the above-described catalyst for the synthesis of an unsaturated aldehyde and an unsaturated carboxylic acid is a process for preparing an extrusion-molded catalyst being characterized in that said catalyst is an extrusion-molded catalyst comprising at least molybdenum, bismuth and iron as metallic elements participating in its catalytic action on the vapor-phase catalytic oxidation reaction in which it is usable to catalyzes the vapor-phase catalytic oxidation reaction for the synthesis of the unsaturated aldehyde and the unsaturated carboxylic acid by using propylene, isobutylene, tert-butyl alcohol or methyl tert-butyl ether as a raw material and using molecular oxygen as an oxygen source; and said process comprising the steps of kneading previously prepared catalyst particles with a liquid medium, and extruding the resulting kneaded material through a predetermined catalyst flow path to form it into a desired shape, wherein a ceramic material is used for at least a part of the catalyst flow path for said extrusion molding step. In this embodiment, it is preferable that in the step of kneading said catalyst particles with a liquid medium, said catalyst particles are kneaded with a liquid medium and a β-1,3-glucan. Further, it is more preferable that in the step of kneading said catalyst particles with a liquid medium, said catalyst particles are kneaded with a liquid medium, a β-1,3-glucan and a cellulose derivative.

There is also provided an invention of use of the above-described catalyst in accordance with the first embodiment of the present invention. That is, in the first embodiment of the present invention, there is provided a process for the synthesis of an unsaturated aldehyde and an unsaturated carboxylic acid being characterized in that in said process, the unsaturated aldehyde and the unsaturated carboxylic acid is synthesized by a vapor-phase catalytic oxidation using propylene, isobutylene, tert-butyl alcohol or methyl tert-butyl ether as a raw material and using molecular oxygen as an oxygen source, and any one of the above-described catalysts in accordance with the first embodiment of the present invention is used as a catalyst for said vapor-phase catalytic oxidation reaction. In particular, preferred is a process for the synthesis of an unsaturated aldehyde and an unsaturated carboxylic acid in which, among the above-described catalysts in accordance with the first embodiment of the present invention, the catalyst prepared by using a ceramic material selected from the group consisting of zirconia, alumina, silica, titania and mixtures of two or more of these materials for at least a part of the catalyst flow path in its extrusion molding step is used as a catalyst for the said vapor-phase catalytic oxidation reaction.

A catalyst for the synthesis of an unsaturated aldehyde and an unsaturated carboxylic acid in accordance with a second embodiment of the present invention comprises an extrusion-molded catalyst being characterized in that said catalyst is an extrusion-molded catalyst comprising at least molybdenum, bismuth and iron as metallic elements participating in its catalytic action on the vapor-phase catalytic oxidation reaction in which it is usable to catalyzes the vapor-phase catalytic oxidation reaction for the synthesis of the unsaturated aldehyde and the unsaturated carboxylic acid by using propylene, isobutylene, tert-butyl alcohol or methyl tert-butyl ether as a raw material and using molecular oxygen as an oxygen source, and said extrusion-molded catalyst being extruded in the step where, when previously prepared catalyst particles containing at least molybdenum, bismuth and iron are subjected to extrusion molding, a kneaded material is prepared by adding a β-1,3-glucan and a liquid to said catalyst particles and kneading the resulting mixture, and then subjected to the extrusion molding. Furthermore, this catalyst may be an extrusion-molded catalyst being extruded in the step where, when previously prepared catalyst particles containing at least molybdenum, bismuth and iron are subjected to extrusion molding, a kneaded material is prepared by adding a β-1,3-glucan, a cellulose derivative and a liquid to the catalyst particles and kneading the resulting mixture, and then subjected to extrusion molding.

In the catalysts in accordance with the second embodiment of the present invention, it is preferable that said liquid be water. Moreover, it is preferable that said β-1,3-glucan be curdlan. On the other hand, it is preferable that said cellulose derivative used in combination with the β-1,3-glucan comprise one or more members selected from the group consisting of methylcellulose, carboxymethylcellulose, hydroxypropyl methylcellulose and hydroxyethyl methylcellulose.

In the second embodiment of the present invention, the process for preparing the above-described catalysts for the synthesis of an unsaturated aldehyde and an unsaturated carboxylic acid may be a process being characterized that said catalyst is an extrusion-molded catalyst comprising at least molybdenum, bismuth and iron as metallic elements participating in its catalytic action on the vapor-phase catalytic oxidation reaction in which it is usable to catalyzes the vapor-phase catalytic oxidation reaction for the synthesis of the unsaturated aldehyde and the unsaturated carboxylic acid by using propylene, isobutylene, tert-butyl alcohol or methyl tert-butyl ether as a raw material and using molecular oxygen as an oxygen source; and said process comprising the steps of adding a β-1,3-glucan and a liquid to previously prepared catalyst particles containing molybdenum, bismuth and iron, and kneading the resulting mixture; and extruding the resulting kneaded material into a desired shape. Alternatively, the process may be comprise the steps of adding a β-1,3-glucan, a cellulose derivative and a liquid to previously prepared catalyst particles containing molybdenum, bismuth and iron, and kneading the resulting mixture; and extrusion-molding the resulting kneaded material into a desired shape.

There is also provided an invention of use of the above-described catalysts in accordance with the second embodiment of the present invention. That is, in the second embodiment of the present invention, there is provided a process for the synthesis of an unsaturated aldehyde and an unsaturated carboxylic acid being characterized in that in said process, the unsaturated aldehyde and the unsaturated carboxylic acid is synthesized by a vapor-phase catalytic oxidation using propylene, isobutylene, tert-butyl alcohol or methyl tert-butyl ether as a raw material and using molecular oxygen as an oxygen source, and any one of the above-described catalysts in accordance with the second embodiment of the present invention is used as a catalyst for said vapor-phase catalytic oxidation reaction. In particular, it is preferable that the above-described catalyst in accordance with the second embodiment of the present invention that is prepared by using water as the liquid used for the preparation of a kneaded material be used as a catalyst for the said vapor-phase catalytic oxidation reaction. Moreover, it is also preferable that the above-described catalyst in accordance with the second embodiment of the present invention that is prepared by using curdlan as the β-1,3-glucan used for the preparation of a kneaded material be used as a catalyst for the said vapor-phase catalytic oxidation reaction. Further, it is more preferable that the above-described catalyst in accordance with the second embodiment of the present invention that is prepared by using one or more members selected from the group consisting of methylcellulose, carboxymethylcellulose, hydroxypropyl methylcellulose and hydroxyethyl methylcellulose as the cellulose derivative used in combination with the β-1,3-glucan be used as a catalyst for the aforesaid vapor-phase catalytic oxidation reaction.

### BEST MODE FOR CARRYING OUT THE INVENTION

The extrusion-molded catalyst of the present invention catalyzes a reaction in which propylene, isobutylene, TBA or MTBE is used as a raw material and a corresponding unsaturated aldehyde and a corresponding unsaturated carboxylic acid is synthesized by vapor-phase catalytic oxidation using molecular oxygen at an oxygen source. More specifically, this is a catalyst suitably usable to synthesize acrolein and acrylic acid, as the corresponding unsaturated aldehyde and unsaturated carboxylic acid, from propylene having 3 carbon atoms, and in a similar manner, when isobutylene, TBA or MTBE is used as a raw material, to synthesize methacrolein and methacrylic acid, as the corresponding unsaturated aldehyde and unsaturated carboxylic acid derived from a branched carbon chain of 4 carbon atoms present in the raw material. Its catalytic action on such vapor-phase catalytic oxidation reactions is achieved by feeding a raw material in gaseous form to the reaction system and effecting reaction between the gaseous molecules of the raw material and molecular oxygen as an oxygen source at catalytic sites present in the surface of the molded catalyst, typically at active sites within pores thereof.

Now, the extrusion-molded catalyst of the present invention and its preparation process are more specifically described below.

The extrusion-molded catalyst of the present invention is an extrusion-molded catalyst comprising at least molybdenum, bismuth and iron as metallic elements participating in its catalytic action. Similarly to conventional extrusion-molded catalysts of this type, it is generally prepared as a molded catalyst having a desired external shape, according to a process comprising:

(1) the step of previously preparing catalyst particles containing predetermined catalyst components;

(2) the step of kneading the resulting catalyst particles (or powders);

(3) the step of extrusion-molding the resulting kneaded material (or kneaded product); and

(4) the step of drying and/or heat-treating thereafter.

In the extrusion-molded catalyst of the present invention, no particular limitation is placed on the step (1) of previously preparing catalyst particles, and there may be employed any of various conventionally known techniques. Usually, in this step (1), an aqueous slurry having a predetermined composition for the catalyst particle, in the present invention, containing at least molybdenum, bismuth and iron in predetermined proportions, as metallic elements participating in its catalytic action, is prepared and then dried to form particles.

No particular limitation is placed on the method for preparing this aqueous slurry containing, as metallic elements participating its catalytic action, at least molybdenum, bismuth and iron in predetermined proportions, provided that it does not cause a significantly localization of the components contained therein. There may be employed any of various techniques which have heretofore been widely used, such as precipitation method and oxide mixing method. In the preparation of the aqueous slurry, various types of compounds, such as oxides, sulfates, nitrates, carbonates, hydroxides, ammonium salts and halides containing the elements to be comprised in the catalyst, may be suitably chosen and used in combination, as raw materials for dissolving the catalyst components in the aqueous medium. For example, raw materials for molybdenum include ammonium paramolybdate, molybdenum trioxide and the like.

No particular limitation is placed on the method for drying the aqueous slurry so prepared to form particles. There may be employed, for example, a drying method using a spray dryer, a drying method using a slurry dryer, a drying method using a drum dryer, or a method comprising evaporating the slurry to dryness and grinding the resulting dry mass to a powder. In view of the advantage that particles can be obtained at the same time as drying and that the resulting particles have a uniform shape and size, it is preferable to form dry spherical particles by means of a spray dryer. The drying conditions may be suitably chosen according to the drying technique employed. For example, when the aqueous slurry is dried by means of a spray dryer, it is desirable to choose the drying conditions so that the inlet temperature of the spray dryer is usually in the range of 100 to 500° C. and the outlet temperature thereof is usually not less than 100° C. and preferably in the range of 105 to 200° C.

In the dry particles obtained by drying the aqueous slurry, as various salts are used as raw materials for catalyst components, the dry particles contain salts (e.g., nitrates) originating from the raw materials and the like. Therefore, they are usually calcined to decompose these salts (e.g., nitrates) to corresponding oxides. However, if the catalyst particles containing such salts as nitrates are extrusion-molded and then calcined to decompose the salts, there is a possibility that the molded product will show a reduction in mechanical strength owing, for example, to the expansion of gas molecules produced by thermal decomposition. In order to avoid this possibility, it is preferable to not only dry the catalyst particles but also calcine them at this stage. No particular limitation is placed on the calcining conditions, and appropriate conditions may be suitably chosen from well-known calcining conditions and employed, depending on the types of the components contained in the aforesaid dry particles. Generally, the calcining temperature used for the particles is chosen so as to be higher than the heating temperature used in the preceding drying step, usually in the range of 200 to 600° C. Depending on the calcining temperature, the calcining time is suitably chosen according to the desired composition of the catalyst to be treated.

In the preparation of particles containing the catalyst components, the shape thereof may vary according to the drying technique, the presence or absence of calcining, the conditions therefor, and the like. However, the shape of the particles can be arbitrarily chosen, so long as it does not interfere with the subsequent extrusion molding and, in particular, the formation of a desired final external shape. For example, when a drying method using a spray dryer is employed, the resulting particles have a spherical external shape. If the average particle size (diameter) thereof is increased, large voids (i.e., large pores) are formed between the particles constituting the molded catalyst after extrusion molding. In many cases, this contributes to an improvement in selectivity. On the other hand, if the average particle size (diameter) is decreased, the number of contact points between particles per unit volume is increased to cause in improvement in the mechanical strength of the resulting molded catalyst. When consideration is taken of the above-described two advantages, in order to achieve a desired improvement in selectivity within an allowable mechanical strength range of the molded catalyst, it is preferable to choose the average particle diameter so as to be in the range of 10 to 150 μm, more preferably 20 to 100 μm, and most preferably 45 to 65 μm.

Next, in the kneading step (2), a predetermined proportion of a liquid (or fluid medium) is mixed with the catalyst particles obtained in step (1), and the resulting mixture is uniformly kneaded. No particular limitation is placed on the apparatus used in the kneading step. For example, there may be used a batch type kneader having double-arm agitating blades, and continuous type kneaders such as axial-rotation reciprocating type and self-cleaning type kneaders. When particle are kneaded with a liquid, the achievement of desired thorough blending (i.e., the end point of kneading) is usually judged by visual observation or hand feeling in many cases. Accordingly, a batch type kneader is preferred for this purpose, because it has the advantage of being able to carry out kneading while monitoring the state of the kneaded material.

As the liquid (or liquid medium) used in the kneading step (2), it is preferable to use a solvent which can be easily removed at the stage of the final molded catalyst and shows a certain or higher affinity and wetting properties for the surface of the catalyst particles. More specifically, it is generally suitable to use water and alcohols. The alcohols which can suitably be used in the kneading step (2) are relatively low-boiling alcohols capable of easily removed by drying, and examples thereof include lower alcohols such as ethanol, methyl alcohol, propyl alcohol and butyl alcohol. Among water and alcohols, it is more preferable to use water, because of its high affinity and wetting properties, its excellent handleability, and its economical efficiency (i.e., a highly pure solvent containing no impurity can be obtained at low cost). With respect to these liquids, not only each of them may be used alone, but also a plurality of mutually miscible liquids may be used in combination. The amount of liquid used may be suitably chosen according to the type and size of the particles, the bulk specific gravity thereof, and further, the type of the liquid. However, it is usually desirable to choose the amount of liquid used so as to be in the range of 10 to 60 parts by mass, more preferably 20 to 50 parts by mass, and most preferably 30 to 45 parts by mass, per 100 parts by mass of the catalyst particles in dried or calcined form.

In the kneading step (2), it is preferable to add a molding aid such as an organic binder, because this molding aid permits the extrusion-molded product to retain its shape and show an improvement in strength. For example, various cellulose derivatives may be used as molding aids, and specific examples thereof include methylcellulose, ethylcellulose, carboxymethylcellulose, carboxymethylcellulose sodium, hydroxyethyl cellulose, hydroxypropyl cellulose, hydroxypropyl methylcellulose, hydroxyethyl methylcellulose, hydroxybutyl methylcellulose, ethylhydroxyethyl cellulose and hydroxypropyl cellulose. Moreover, there may be added conventionally known additives that are effective in enhancing the mechanical strength of extrusion-molded catalysts of this type. Such additives include, for example, inorganic compounds such as graphite and diatomaceous earth; and inorganic fibers such as glass fiber, ceramic fiber and carbon fiber.

In the extrusion-molded catalyst in accordance with the first embodiment of the present invention, when a kneaded material is prepared by adding a liquid (or liquid medium) to the catalyst particles in the kneading step (2), a small amount of a β-1,3-glucan is added to the kneaded material. Preferably, besides the β-1,3-glucan, any of the aforesaid various cellulose derivatives which can be used as molding aids may be added to the kneaded material. Although no particular limitation is placed on the origin of the β-1,3-glucans which can be used, those of microbial, vegetable and animal origin can preferably be used. Similarly to various cellulose derivatives which can be used as molding aids, β-1,3-glucans, when added to the kneaded material, bring about an improvement in molding properties at the time of extrusion molding.

Moreover, β-1,3-glucans have water-retaining properties. When water or an alcohol is used as the liquid (or liquid medium) in the preparation of a kneaded material, the molded product obtained by extrusion-molded the resulting kneaded material can contain a larger amount of water or the alcohol without detracting from its moldability. Consequently, desirable pores are developed in the final catalyst obtained by subjecting the molded product containing a larger amount of water or the alcohol to the drying and/or heat-treatment step (4) which will be described later, resulting in the preparation of a catalyst having higher selectivity. That is, by adding a small amount of a β-1,3-glucan during the preparation of a kneaded material, desirable pores are developed in the final catalyst to yield a catalyst having high catalytic activity and high selectivity for an unsaturated aldehyde and an unsaturated carboxylic acid. This effect of the addition of a β-1,3-glucan becomes more pronounced when water is used as the liquid used in the kneading step. For example, when water is used, the addition of a β-1,3-glucan makes it possible to increase the upper limit of the amount of liquid used to 70 parts by mass per 100 parts by mass of the catalyst particles.

The β-1,3-glucans which can suitably be used include, for example, curdlan, laminaran, paramylon, callose, pachyman and scleroglucan. Among others, the β-1,3-glucans of microbial origin are preferably used in the present invention. Specifically, curdlan, paramylon and the like are preferred, and curdlan is especially preferred. These β-1,3-glucans may be used alone or in admixture of two or more. Although β-1,3-glucans may be used in an unpurified or purified state, the presence of large amounts of metals and ignition residues arising from impurities in unpurified β-1,3-glucans may cause a reduction in catalyst performance. Accordingly, it is preferable that the content of impurities in the β-1,3-glucan used be as low as possible.

The amount of β-1,3-glucan added in the kneading step (2) may be suitably chosen according to the type and size of the catalyst particles, the type of the liquid, and the like. However, its amount is usually chosen so as to be in the range of 0.05 to 15 parts by mass, preferably not less than 0.1 part by mass and not greater than 10 parts by mass, per 100 parts by mass of the catalyst particles obtained in step (1). As the amount of β-1,3-glucan added is increased, the moldability of the resulting kneaded material tends to be improved. On the other hand, as its amount added is decreased, the after-treatment step (4) carried out after extrusion molding, such as drying and/or heat treatment, tends to become simpler.

In the kneading step (2), besides the addition of the β-1,3-glucan, a molding aid may be added as described above. In the present invention, the use of a cellulose derivative as a molding aid in addition to the aforesaid β-1,3-glucan is effective in yielding a catalyst having higher activity and selectivity.

As the cellulose derivative used in combination with the β-1,3-glucan, there may be used any of the above-enumerated various cellulose derivatives. Among others, methylcellulose, carboxymethylcellulose, hydroxypropyl methylcellulose and hydroxyethyl methylcellulose are preferred. For this purpose, these cellulose derivatives may be used alone or in admixture of two or more. From the viewpoint of function as a molding aid, a cellulose derivative having a viscosity in the range of 1,000 to 10,000 Pam·s as measured at 20° C. for its 2% aqueous solution is more preferred because it can provide better moldability.

The amount of cellulose derivative added in combination with the β-1,3-glucan may be suitably chosen according to the type and size of the catalyst particles, the type of the liquid, and the like. However, its amount is usually chosen so as to be in the range of 0.05 to 15 parts by mass, preferably not less than 0.1 part by mass and not greater than 10 parts by mass, per 100 parts by mass of the catalyst particles obtained in step (1). As the amount of cellulose derivative added is increased, the moldability of the resulting kneaded material tends to be improved. On the other hand, as its amount added is decreased, the after-treatment step (4) carried out after extrusion molding, such as drying and/or heat treatment, tends to become simpler.

In the first embodiment of the present invention, when both a β-1,3-glucan and a cellulose derivative are used in the kneading step (2), it is usually preferable to choose their combined amount added so as to be not less than 0.1 part by mass and not greater than 20 parts by mass, per 100 parts by mass of the catalyst particles obtained in step (1).

Moreover, in the first embodiment of the present invention, when both a β-1,3-glucan and a cellulose derivative are used in the kneading step (2), it is preferable to choose the ratio between their amounts added so that the cellulose derivative is used in an amount of not greater than 30 parts by mass, more preferably not greater than 6 parts by mass, for 1 part by mass of the β-1,3-glucan. Also in the second embodiment of the present invention which will be described below, when both a β-1,3-glucan and a cellulose derivative are used in the kneading step (2), it is preferable to choose the ratio between their amounts added in the same manner as described above.

In the extrusion molding step (3), the kneaded material obtained in the kneading step (2) is subjected to extrusion molding. Although no particular limitation is placed on the apparatus used for extrusion molding, there may be used, for example, an auger type extruder or a piston type extruder. The kneading step (2) and the extrusion molding step (3) may be carried out continuously, and may hence be carried out simultaneously by using an integral apparatus adapted for this purpose.

In the extrusion-molded catalyst in accordance with the second embodiment of the present invention, its extrusion molding is carried out by using a ceramic material for at least a part of the catalyst flow path with which the kneaded material (or kneaded product) comes into contact under pressure in the extrusion molding step. When a ceramic material is used for at least a part of the catalyst flow path, the final extrusion-molded catalyst has more desirable pores developed therein and exhibits higher catalytic activity and higher selectivity for an unsaturated aldehyde and an unsaturated carboxylic acid, as compared with the case where a conventional catalyst flow path formed entirely of metal (carbon steel or tool steel). As the proportion of the ceramic material used in the surface of the catalyst flow path is increased, its effects become more pronounced.

As used herein, the term “catalyst flow path” means a flow path which extends from the end of the extruder to the catalyst outlet of the extrusion molding die and with which the kneaded material (or kneaded product) pressurized for extrusion purposes comes into direct contact. In the present invention, there is used a catalyst flow path in which at least a part of the catalyst flow path surface coming into contact with the catalyst particles contained in the kneaded material (or kneaded product) is formed of a ceramic material. For example, the catalyst flow path itself may be formed of a ceramic material. Alternatively, there may also be used a catalyst flow path comprising a member made of metal (carbon steel or tool steel) and provided with a surface coating layer of ceramic material (i.e., a ceramic layer) formed thereon. This ceramic layer may be provided by forming a sintered ceramic layer having a thickness of not less than 0.05 mm and preferably not less than 0.5 mm and attaching it to the main body of the die by shrink fitting, adhesive bonding, caulking or the like; or by thermally spraying a ceramic material onto the main body of the die so as to give a thickness of not less than 0.05 mm and preferably not less than 0.5 mm. Where the small size of the die makes it difficult to coat it with a ceramic layer as described above, the die parts or the whole die may be formed of a ceramic material.

No particular limitation is placed on the ceramic material used in the present invention, for example, as a coating layer for the surface of the catalyst flow path, provided that the metallic elements constituting the ceramic material may be added to the desired molded catalyst. For example, there may be used nitrides, carbides, carbonitrides and oxides of metals such as B, Si, Ti, V, Cr, Zr, W and Al. Among these, oxides such as zirconia, alumina, silica and titania are especially preferred. Of the four enumerated ceramic materials, zirconia is most preferred. When zirconia is used, it is more preferably used in the form of a so-called. “partially stabilized zirconia” containing a stabilizer such as yttria, calcia, ceria or magnesia.

The extrusion-molded material is cut to an appropriate length. No particular is placed on the shape of the extrusion-molded material, and it may have any of various shapes such as rings (or cylinders), columns and stellate pillars.

In the drying/calcining step (4), the extrusion-molded material obtained in the extrusion molding step is first dried to obtain a dried molded product. No particular limitation is placed on the drying method employed in this step, provided that the liquid (or fluid medium) remaining after the extrusion molding of the kneaded material (or kneaded product) can be removed by evaporation. For example, there may be employed any of well-known drying methods such as hot-air drying, humidity drying, far-infrared drying and microwave drying. Among the above-enumerated drying methods, a single means may be employed or a plurality of techniques may be suitably employed in combination. Depending on the liquid (or fluid medium) to be removed by evaporation in this drying step, the drying conditions may be suitably chosen according to the desired content of the liquid (or fluid medium) remaining after drying [for example, the desired water content when the aforesaid liquid (or fluid medium) is water].

Usually, the dried molded product obtained by this drying treatment is further subjected to a calcining treatment. This calcining step may be carried out, for example, in order to remove the added molding aids (e.g., an organic binder) by thermal decomposition and in order to calcine the catalyst particles. Where the catalyst particles formed in the previously described step (1) are previously calcined particles, further calcining may be omitted because they no longer contain components to be thermally decomposed by calcining. Although the temperature for the calcining carried out in step (4) should be chosen so as to meet its purpose, it is usually chosen so as to be in the range of 200 to 600° C.

In the extrusion-molded catalyst of the present invention, even when only one of the two means comprising the above-described feature of the first embodiment [i.e., a β-1,3-glucan is added to the kneaded material prepared in the kneading step (2)] and the above-described feature of the second embodiment [i.e., in the extrusion molding step (3), a ceramic material is used for at least a part of the catalyst flow path with which the kneaded material (or kneaded product) comes into contact under pressure] is employed, sufficiently desirable pores are developed in the final extrusion-molded catalyst to yield a catalyst having high catalytic activity and high selectivity for an unsaturated aldehyde and an unsaturated carboxylic acid. Moreover, when both of these two means are employed in the extrusion-molded catalyst of the present invention, more desirable pores are developed in the finally obtained extrusion-molded catalyst to yield a catalyst having higher catalytic activity and higher selectivity for an unsaturated aldehyde and an unsaturated carboxylic acid. When both of these two means are employed, these means are associated with the kneading step (2) and the extrusion molding step (3), respectively. Consequently, operating conditions suitable therefor may be chosen separately, so that a more preferable extrusion-molded catalyst can be obtained.

The catalyst of the present invention, which is prepared as an extrusion-molded catalyst according to the preparation process of the present invention, is one comprising at least molybdenum, bismuth and iron as metallic elements participating in its catalytic action. However, it may also comprise additional elements such as silicon, cobalt, nickel, chromium, lead, manganese, calcium, magnesium, niobium, silver, barium, tin, tantalum, zinc, phosphorus, boron, sulfur, selenium, tellurium, cerium, tungsten, antimony, titanium, lithium, sodium, potassium, rubidium, cesium and thallium. More specifically, the catalyst of the present invention is preferably prepared as a catalyst having an average composition represented by the following general formula.

MoₐBi_{b}Fe_{c}M_{d}XₑY_{f}Z_{g}SiₕOᵢ

wherein Mo, Bi, Fe, Si and O represent molybdenum, bismuth, iron, silicon and oxygen, respectively;

M represents at least one element selected from the group consisting of cobalt and nickel;

X represents at least one element selected from the group consisting of chromium, lead, manganese, calcium, magnesium, niobium, silver, barium, tin, tantalum and zinc;

Y represents at least one element selected from the group consisting of phosphorus, boron, sulfur, selenium, tellurium, cerium, tungsten, antimony and titanium; and

Z represents at least one element selected from the group consisting of lithium, sodium, potassium, rubidium, cesium and thallium.

Moreover, a, b, c, d, e, f, g, h and i represent the atomic ratios of the aforesaid elements. For a=12, they may be chosen so that b=0.01-3, c=0.01-5, d=1-12, e=0-8, f=0-5, g=0.001-2 and h=0-20. i is the atomic ratio of oxygen which, at the atomic ratios of the foregoing elements, is required to satisfy the valence of each constituent element.

In the process for the synthesis of an unsaturated aldehyde and an unsaturated carboxylic acid in accordance with the present invention, the extrusion-molded catalyst of the present invention, which is conveniently prepared according to the preparation process of the present invention, is used. Thus, a raw material comprising propylene, isobutylene, TBA or MTBE is subjected to a vapor-phase catalytic oxidation reaction using molecular oxygen at an oxygen source, and thereby converted to an unsaturated aldehyde and an unsaturated carboxylic acid which have a corresponding carbon chain. This vapor-phase catalytic oxidation reaction is carried out by charging the extrusion-molded catalyst into a reaction tube and passing therethrough a mixed gas containing a raw material comprising propylene, isobutylene, TBA or MTBE and an oxygen source comprising molecular oxygen used in a predetermined proportion to the raw material. In the aforesaid reaction tube, the catalyst of the present invention may be charged in a state diluted with an inert carrier such as silica, alumina, silica-alumina, silicon carbide, titania, magnesia, ceramic balls or stainless steel.

As molecular oxygen used as an oxygen source, it is economical to use a gaseous mixture of molecular oxygen and molecular nitrogen (e.g., air). However, if it is necessary to raise the partial pressure of oxygen according to the reaction conditions, air enriched with pure oxygen may be used. The molar ratio between raw material molecules and oxygen molecules present in the mixed gas fed into the reaction tube may vary according to the reaction conditions. However, in order to enhance the yields of an unsaturated aldehyde and an unsaturated carboxylic acid, the molar ratio is preferably chosen so as to range from 1:0.5 to 1:3. It is preferable that the mixed gas to be fed into the reaction tube comprise water vapor in addition to gaseous raw material molecules and molecular oxygen. It is also preferable that the mixed gas be diluted with an inert gas. As the aforesaid inert gas, there may be used any general-purpose inert gas that shows no reactivity with the raw material and the unsaturated aldehyde and unsaturated carboxylic acid being desired product, such as nitrogen or carbon dioxide. When water vapor is added, it is desirable that the content of water vapor in the mixed gas fed into the reaction tube be not greater than 45% by volume (for example, in the range of 1 to 45% by volume).

Accordingly, the content of the raw material (i.e., propylene, isobutylene, TBA or MTBE) in the mixed gas fed into the reaction tube also depends on the amounts of the aforesaid inert gas and water vapor added and may vary widely. However, it is preferable to choose the content of the raw material so as to be, for example, in the range of 1 to 20% by volume.

Moreover, it is preferable that the reaction pressure be chosen so as to range from atmospheric pressure to several hundred kPa. It is also desirable to choose the reaction pressure so as to give a proper average residence time (or contact time). The reaction temperature may generally be chosen so as to be in the range of 200 to 450° C. However, it is especially preferable to choose the reaction temperature so as to be in the range of 250 to 400° C. The aforesaid reaction is usually carried out in a fixed bed. In this case, the catalyst bed may consist of a single catalyst layer or two or more catalyst layers, depending on the average residence time (or contact time) in each layer. Although the overall contact time may be suitably chosen according to the reaction pressure, the reaction temperature, and the degree of dilution with an inert gas, it is usually preferable to choose the overall contact time so as to be in the range of 1.5 to 15 seconds.

The process for the synthesis of an unsaturated aldehyde and an unsaturated carboxylic acid in accordance with the present invention may be carried out in an embodiment in which, depending on the catalyst composition used and the reaction conditions employed, only one of the unsaturated aldehyde and the unsaturated carboxylic acid is selectively obtained as the desired product, and the present invention also comprehends such embodiments. For example, the present invention comprehends an embodiment in which the formation of undesired by-products other than the unsaturated aldehyde and the unsaturated carboxylic acid is suppressed, whereas the vapor-phase oxidation reaction for producing the desired products is limited to the formation of the unsaturated aldehyde and does not get to the formation of the unsaturated carboxylic acid.

### EXAMPLES

The present invention is more specifically explained with reference to the following examples and comparative examples. Although these examples are typical of the best embodiments of the present invention, the present invention is not limited by these modes of examples. In the description of the examples and the comparative examples, the term “parts” refers to parts by mass, and a batch type kneaded equipped with double-arm agitating blades was used in the kneading step. The composition of the mixed gas fed into the reaction tube and containing the raw material and the composition of the gas discharged from the reaction tube and containing the products were analyzed by gas chromatography.

In the examples and the comparative examples, the degree of conversion of the raw material (olefin, TBA or MTBE) (hereinafter referred to as the ratio of conversion), and the selectivity for the unsaturated aldehyde or unsaturated carboxylic acid formed were calculated according to the following formulas.

Ratio of conversion (%)=A/B×100

Selectivity for unsaturated aldehyde (%)=C/A×100

Selectivity for unsaturated carboxylic acid (%)=D/A×100

where A is the number of moles of the raw material (olefin, TBA or MTBE) which underwent a reaction in the reaction tube and was converted to another molecule;

B is the number of moles of the raw material (olefin, TBA or MTBE) fed into the reaction tube;

C is the number of moles of the unsaturated aldehyde contained in the gas discharged from the reaction gas; and

D is the number of moles of the unsaturated carboxylic acid contained in the gas discharged from the reaction gas.

### Example A-1

Five hundred (500) parts of ammonium paramolybdate, 6.2 parts of ammonium paratungstate, 1.4 parts of potassium nitrate, 27.5 parts of antimony trioxide and 55.0 parts of bismuth trioxide were added to 1,000 parts of purified water, and this mixture was heated with stirring (fluid A). Separately, 114.4 parts of ferric nitrate, 295.3 parts of cobalt nitrate and 35.1 parts of zinc nitrate were successively added to 1,000 parts of purified water and dissolved therein (fluid B). After an aqueous slurry was prepared by adding fluid B to fluid A, this aqueous slurry was formed into dry spherical particles having an average particle diameter of 60 μm by means of a spray dryer. These dry spherical particles were calcined at 300° C. for 1 hour to form a calcined catalyst material.

To 500 parts of the calcined catalyst material thus obtained was added 25 parts of curdlan, followed by dry blending. After 160 parts of purified water was mixed therewith, the resulting mixture was blended (kneaded) on a kneader until a clayish material was obtained. Thereafter, using an auger type extruder, the clayish material was extrusion-molded to obtain a molded catalyst in the form of pieces having an outer diameter of 5 mm, an inner diameter of 2 mm and a length of 5 mm.

Then, using a hot-air dryer, the resulting molded catalyst was dried at 110° C. to obtain a dried molded catalyst. Thereafter, this molded catalyst was calcined again at 510° C. for 3 hours to obtain a finally calcined molded catalyst.

The composition of the elements, except oxygen (hereinafter the same), constituting the molded catalyst thus obtained was as follows:

Mo₁₂Wo_{0.1}Bi_{1.0}Fe_{1.2}Sb_{0.8}Co_{4.3}Zn_{0.5}K_{0.06}

This molded catalyst was charged into a reaction tube made of stainless steel, and a raw material gas comprising 5% of propylene, 12% of oxygen, 10% of water vapor and 73% of nitrogen (on a volume percentage basis) was reacted therein at atmospheric pressure under conditions including a contact time of 3.6 seconds and a reaction temperature of 310° C. As a result of the reaction, the ratio of conversion of propylene was 99.0%, the selectivity for acrolein was 91.1%, and the selectivity for acrylic acid was 6.5%. The amount of by-products other than the desired products was 2.4%.

### Example A-2

A molded catalyst was prepared under the similar conditions to those in Example A-1, except that the preparation conditions of Example A-1 were modified by adding 5 parts of curdlan and 25 parts of methylcellulose in place of 25 parts of curdlan. Using the molded catalyst thus obtained, a vapor-phase catalytic oxidation reaction was carried out under the same conditions as in Example A-1. As a result of the reaction, the ratio of conversion of propylene was 99.0%, the selectivity for acrolein was 91.1%, and the selectivity for acrylic acid was 6.6%. The amount of by-products other than the desired products was 2.3%.

### Comparative Example A-1

A molded catalyst was prepared under the similar conditions to those in Example A-1, except that the preparation conditions of Example A-1 were modified by adding 160 parts of purified water alone to 500 parts of the calcined catalyst material without the addition of curdlan, and kneading the resulting mixture. The molded catalyst thus obtained had very low shape retention properties. Using the molded catalyst thus obtained, a vapor-phase catalytic oxidation reaction was carried out under the same conditions as in Example A-1. As a result of the reaction, the ratio of conversion of propylene was 98.6%, the selectivity for acrolein was 87.0%, and the selectivity for acrylic acid was 6.1%. The amount of by-products other than the desired products was 6.9%.

### Comparative Example A-2

A molded catalyst was prepared under the similar conditions to those in Example A-1, except that the preparation conditions of Example A-1 were modified by adding 25 parts of methylcellulose in place of 25 parts of curdlan. Using the molded catalyst thus obtained, a vapor-phase catalytic oxidation reaction was carried out under the same conditions as in Example A-1. As a result of the reaction, the ratio of conversion of propylene was 98.9%, the selectivity for acrolein was 90.4%, and the selectivity for acrylic acid was 6.2%. The amount of by-products other than the desired products was 3.4%.

### Example A-3

Five hundred (500) parts of ammonium paramolybdate, 6.2 parts of ammonium paratungstate, 23.0 parts of cesium nitrate, 24.0 parts of antimony trioxide and 33.0 parts of bismuth trioxide were added to 1,000 parts of purified water, and this mixture was heated with stirring (fluid A). Separately, 190.7 parts of ferric nitrate, 75.5 parts of nickel nitrate, 453.3 parts of cobalt nitrate, 31.3 parts of lead nitrate and 2.8 parts of 85% phosphoric acid were successively added to 1,000 parts of purified water and dissolved therein (fluid B). After an aqueous slurry was prepared by adding fluid B to fluid A, this aqueous slurry was formed into dry spherical particles having an average particle diameter of 60 μm by means of a spray dryer. These dry spherical particles were calcined at 300° C. for 1 hour and then at 510° C. for 3 hours to form a calcined catalyst material.

To 500 parts of the calcined catalyst material thus obtained was added 20 parts of curdlan, followed by dry blending. After 160 parts of purified water was mixed therewith, the resulting mixture was blended (kneaded) on a kneader until a clayish material was obtained. Thereafter, using a piston type extruder, the clayish material was extrusion-molded to obtain a molded catalyst in the form of pieces having an outer diameter of 5 mm, an inner diameter of 2 mm and a length of 5 mm.

Then, using a hot-air dryer, the resulting molded catalyst was dried at 110° C. to obtain a dried molded catalyst. Thereafter, this molded catalyst was calcined again at 400° C. for 3 hours to obtain a finally calcined molded catalyst.

The composition of the elements constituting the molded catalyst thus obtained was as follows:

Mo₁₂W_{0.1}Bi_{0.6}Fe₂Sb_{0.7}Ni_{1.1}Co_{6.6}Pb_{0.4}P_{0.1}Cs_{0.5}

This molded catalyst was charged into a reaction tube made of stainless steel, and a raw material gas comprising 5% of isobutylene, 12% of oxygen, 10% of water vapor and 73% of nitrogen (on a volume percentage basis) was reacted therein at atmospheric pressure under conditions including a contact time of 3.6 seconds and a reaction temperature of 340° C. As a result of the reaction, the ratio of conversion of isobutylene was 97.9%, the selectivity for methacrolein was 89.9%, and the selectivity for methacrylic acid was 3.9%. The amount of by-products other than the desired products was 6.2%.

### Example A-4

A molded catalyst was prepared under the similar conditions to those in Example A-3, except that the preparation conditions of Example A-3 were modified by adding 5 parts of curdlan and 15 parts of methylcellulose in place of 20 parts of curdlan. Using the molded catalyst thus obtained, a vapor-phase catalytic oxidation reaction was carried out under the same conditions as in Example A-3. As a result of the reaction, the ratio of conversion of isobutylene was 97.9%, the selectivity for methacrolein was 89.9%, and the selectivity for methacrylic acid was 4.1%. The amount of by-products other than the desired products was 6.1%.

### Example A-5

A molded catalyst was prepared under the similar conditions to those in Example A-3, except that the preparation conditions of Example A-3 were modified by adding 5 parts of curdlan and 20 parts of methylcellulose in place of 20 parts of curdlan. Using the molded catalyst thus obtained, a vapor-phase catalytic oxidation reaction was carried out under the same conditions as in Example A-3. As a result of the reaction, the ratio of conversion of isobutylene was 98.0%, the selectivity for methacrolein was 89.9%, and the selectivity for methacrylic acid was 4.0%. The amount of by-products other than the desired products was 6.1%.

### Example A-6

A molded catalyst was prepared under the similar conditions to those in Example A-3, except that the preparation conditions of Example A-3 were modified by adding 5 parts of curdlan and 20 parts of hydroxypropyl methylcellulose in place of 20 parts of curdlan. Using the molded catalyst thus obtained, a vapor-phase catalytic oxidation reaction was carried out under the same conditions as in Example A-3. As a result of the reaction, the ratio of conversion of isobutylene was 98.2%, the selectivity for methacrolein was 89.9%, and the selectivity for methacrylic acid was 4.0%. The amount of by-products other than the desired products was 6.1%.

### Comparative Example A-3

A molded catalyst was prepared under the similar conditions to those in Example A-3, except that the preparation conditions of Example A-3 were modified by adding 20 parts of methylcellulose in place of 20 parts of curdlan. Using the molded catalyst thus obtained, a vapor-phase catalytic oxidation reaction was carried out under the same conditions as in Example A-3. As a result of the reaction, the ratio of conversion of isobutylene was 97.5%, the selectivity for methacrolein was 89.5%, and the selectivity for methacrylic acid was 3.5%. The amount of by-products other than the desired products was 7.0%.

### Example B-1

Five hundred (500) parts of ammonium paramolybdate, 6.2 parts of ammonium paratungstate, 1.4 parts of potassium nitrate, 27.5 parts of antimony trioxide and 60.5 parts of bismuth trioxide were added to 1,000 parts of purified water, and this mixture was heated with stirring (fluid A). Separately, 114.4 parts of ferric nitrate, 295.3 parts of cobalt nitrate and 35.1 parts of zinc nitrate were successively added to 1,000 parts of purified water and dissolved therein (fluid B). After an aqueous slurry was prepared by adding fluid B to fluid A, this uniformly mixed aqueous slurry was dried with a spray dryer to form dry spherical particles having an average particle diameter of 60 μm. These dry spherical particles were calcined at 300° C. for 1 hour to obtain a calcined particulate catalyst material.

To 500 parts of the calcined particulate catalyst material thus obtained was added 15 parts of methylcellulose, followed by dry blending. After 160 parts of purified water was added to this dry blend and mixed therewith, the resulting mixture was blended (kneaded) on a kneader until a clayish material was obtained. Thereafter, using an auger type extruder, the kneaded material was extrusion-molded to obtain a molded catalyst in the form of cylindrical pieces having an outer diameter of 5 mm, an inner diameter of 2 mm and an average length of 5 mm. As the die members for this extrusion molding, there were used an inner die (core) formed of 3Al₂O₃·2SiO₂ and an outer die formed by bonding an about 2 mm thick layer of 3Al₂O₃·2SiO₂ to the surface of carbon steel (S45C).

Then, using a hot-air dryer, the resulting molded catalyst was dried at 110° C. to obtain a dried molded catalyst. Moreover, this dried molded catalyst was calcined again at 510° C. for 3 hours to obtain a finally calcined molded catalyst.

The composition of the elements, except oxygen (hereinafter the same), constituting the finally calcined molded catalyst thus obtained was as follows:

Mo₁₂W_{0.1}Bi_{1.1}Fe_{1.2}Sb_{0.8}Co_{4.3}Zn_{0.5}K_{0.06}

This finally calcined molded catalyst was charged into a reaction tube made of stainless steel, and a mixed raw material gas comprising 5% of propylene, 12% of oxygen, 10% of water vapor and 73% of nitrogen (on a volume percentage basis) was passed through the charged catalyst layer with a contact time of 3.6 seconds and thereby reacted at a temperature of 310° C. under atmospheric pressure. As a result of this vapor-phase catalytic oxidation reaction, the ratio of conversion of the raw material, propylene, was 99.0%, the selectivity for the recovered acrolein was 91.1%, and the selectivity for the recovered acrylic acid was 6.6%. Accordingly, the combined yield of acrolein and acrylic acid was found to be 96.7%.

### Example B-2

A finally calcined molded catalyst was prepared under the similar conditions and according to the similar procedure to those in Example B-1, except that the steps and conditions for preparing a finally calcined molded catalyst as described in Example B-1 were modified by using, as the die members for extrusion molding, an inner die (core) formed of 3Al₂O₃·2SiO₂ and an outer die formed of carbon steel (S45C). Using the finally calcined molded catalyst thus obtained, a vapor-phase catalytic oxidation reaction was carried out under the same reaction conditions as described in Example B-1.

As a result of the reaction using the finally calcined molded catalyst prepared in this Example B-2, the ratio of conversion of the raw material, propylene, was 98.9%, the selectivity for the recovered acrolein was 90.9%, and the selectivity for the recovered acrylic acid was 6.5%. Accordingly, the combined yield of acrolein and acrylic acid was found to be 96.3%.

### Comparative Example B-1

A finally calcined molded catalyst was prepared under the similar conditions and according to the similar procedure to those in Example B-1, except that the steps and conditions for preparing a finally calcined molded catalyst as described in Example B-1 were modified by using, as the die members for extrusion molding, an inner die (core) and an outer die which were both formed of carbon steel (S45C). Using the finally calcined molded catalyst thus obtained, a vapor-phase catalytic oxidation reaction was carried out under the same reaction conditions as described in Example B-1.

As a result of the reaction using the finally calcined molded catalyst prepared in this Comparative Example B-1, the ratio of conversion of the raw material, propylene, was 98.6%, the selectivity for the recovered acrolein was 90.2%, and the selectivity for the recovered acrylic acid was 6.1%. Accordingly, the combined yield of acrolein and acrylic acid was found to be 95.0%.

### Example B-3

Five hundred (500) parts of ammonium paramolybdate, 6.2 parts of ammonium paratungstate, 23.0 parts of cesium nitrate, 27.4 parts of antimony trioxide and 33.0 parts of bismuth trioxide were added to 1,000 parts of purified water, and this mixture was heated with stirring (fluid A). Separately, 190.7 parts of ferric nitrate, 75.5 parts of nickel nitrate, 446.4 parts of cobalt nitrate, 31.3 parts of lead nitrate and 2.8 parts of 85% phosphoric acid were successively added to 1,000 parts of purified water and dissolved therein (fluid B). After an aqueous slurry was prepared by adding fluid B to fluid A, this uniformly mixed aqueous slurry was dried with a spray dryer to form dry spherical particles having an average particle diameter of 60 μm. These dry spherical particles were calcined at 300° C. for 1 hour and at 510° C. for 3 hours to obtain a calcined particulate catalyst material.

To 500 parts of the calcined particulate catalyst material thus obtained was added 20 parts of methylcellulose, followed by dry blending. After 160 parts of purified water was added to this dry blend and mixed therewith, the resulting mixture was blended (kneaded) on a kneader until a clayish material was obtained. Thereafter, using a piston type extruder, the kneaded material was extrusion-molded to obtain a molded catalyst in the form of pieces having an outer diameter of 5 mm, an inner diameter of 2 mm and an average length of 5 mm. As the die members for this extrusion molding, there were used an inner die (core) formed of partially yttria-stabilized zirconia and an outer die formed by bonding an about 1 cm thick layer of partially yttria-stabilized zirconia to the surface of tool steel (SKD61).

Then, using a hot-air dryer, the resulting molded catalyst was dried at 110° C. to obtain a dried molded catalyst. Thereafter, this dried molded catalyst was calcined again at 400° C. for 3 hours to obtain a finally calcined molded catalyst.

The composition of the elements, except oxygen, constituting the finally calcined molded catalyst thus obtained was as follows:

Mo₁₂W_{0.1}Bi_{0.6}Fe₂Sb_{0.8}Ni_{1.1}Co_{6.5}Pb_{0.4}P_{0.1}Cs_{0.5}

This finally calcined molded catalyst was charged into a reaction tube made of stainless steel, and a mixed raw material gas comprising 5% of isobutylene, 12% of oxygen, 10% of water vapor and 73% of nitrogen (on a volume percentage basis) was passed through the charged catalyst layer with a contact time of 3.6 seconds and thereby reacted at a temperature of 340° C. under atmospheric pressure. As a result of this vapor-phase catalytic oxidation reaction, the ratio of conversion of the raw material, isobutylene, was 98.0%, the selectivity for the recovered methacrolein was 89.9%, and the selectivity for the recovered methacrylic acid was 4.0%. Accordingly, the combined yield of methacrolein and methacrylic acid was found to be 92.0%.

### Example B-4

A finally calcined molded catalyst was prepared under the similar conditions and according to the similar procedure to those in Example B-3, except that the steps and conditions for preparing a finally calcined molded catalyst as described in Example B-3 were modified by using, as the die members for extrusion molding, an inner die (core) formed of partially yttria-stabilized zirconia and an outer die formed of tool steel (SKD61). Using the finally calcined molded catalyst thus obtained, a vapor-phase catalytic oxidation reaction was carried out under the same reaction conditions as described in Example B-3.

As a result of the reaction using the finally calcined molded catalyst prepared in this Example B-4, the ratio of conversion of the raw material, isobutylene, was 97.9%, the selectivity for the recovered methacrolein was 89.7%, and the selectivity for the recovered methacrylic acid was 3.9%. Accordingly, the combined yield of methacrolein and methacrylic acid was found to be 91.6%.

### Comparative Example B-2

A finally calcined molded catalyst was prepared under the similar conditions and according to the similar procedure to those in Example B-3, except that the steps and conditions for preparing a finally calcined molded catalyst as described in Example B-3 were modified by using, as the die members for extrusion molding, an inner die (core) and an outer die which were both formed of tool steel (SKD61). Using the finally calcined molded catalyst thus obtained, a vapor-phase catalytic oxidation reaction was carried out under the same reaction conditions as described in Example B-3.

As a result of the reaction using the finally calcined molded catalyst prepared in this Comparative Example B-2, the ratio of conversion of the raw material, isobutylene, was 97.2%, the selectivity for the recovered methacrolein was 89.4%, and the selectivity for the recovered methacrylic acid was 3.4%. Accordingly, the combined yield of methacrolein and methacrylic acid was found to be 90.2%.

### Example B-5

Using the catalyst of Example B-3, a reaction was carried out in the similar manner to Example B-3, except that, as the raw material, TBA was used in place of isobutylene.

The finally calcined molded catalyst of Example B-3 was charged into a reaction tube made of stainless steel, and a mixed raw material gas comprising 5% of TBA, 12% of oxygen, 10% of water vapor and 73% of nitrogen (on a volume percentage basis) was passed through the charged catalyst layer with a contact time of 3.6 seconds and thereby reacted at a temperature of 340° C. under atmospheric pressure. As a result of this vapor-phase catalytic oxidation reaction, the ratio of conversion of the raw material, TBA, was 100.0%, the selectivity for the recovered methacrolein was 88.8%, and the selectivity for the recovered methacrylic acid was 3.1%. Accordingly, the combined yield of methacrolein and methacrylic acid was found to be 91.8%.

### Comparative Example B-3

Using the catalyst of Comparative Example B-2, a reaction was carried out in the similar manner to Comparative Example B-2, except that, as the raw material, TBA was used in place of isobutylene.

The finally calcined molded catalyst of Comparative Example B-2 was charged into a reaction tube made of stainless steel, and a mixed raw material gas comprising 5% of TBA, 12% of oxygen, 10% of water vapor and 73% of nitrogen (on a volume percentage basis) was passed through the charged catalyst layer with a contact time of 3.6 seconds and thereby reacted at a temperature of 340° C. under atmospheric pressure. As a result of this vapor-phase catalytic oxidation reaction, the ratio of conversion of the raw material, TBA, was 100.0%, the selectivity for the recovered methacrolein was 88.2%, and the selectivity for the recovered methacrylic acid was 2.5%. Accordingly, the combined yield of methacrolein and methacrylic acid was found to be 90.7%.

### Example B-6

A finally calcined molded catalyst was prepared under the similar conditions and according to the similar procedure to those in Example B-3, except that the steps and conditions for preparing a finally calcined molded catalyst as described in Example B-3 were modified by using 20 parts of curdlan in place of 20 parts of methylcellulose. Using the finally calcined molded catalyst thus obtained, a vapor-phase catalytic oxidation reaction was carried out under the same reaction conditions as described in Example B-3.

As a result of the reaction using the finally calcined molded catalyst prepared in this Example B-6, the ratio of conversion of the raw material, isobutylene, was 98.1%, the selectivity for the recovered methacrolein was 89.9%, and the selectivity for the recovered methacrylic acid was 4.1%. Accordingly, the combined yield of methacrolein and methacrylic acid was found to be 92.2%.

The catalyst for the synthesis of an unsaturated aldehyde and an unsaturated carboxylic acid in accordance with the present invention is an extrusion-molded catalyst containing at least molybdenum, bismuth and iron as metallic elements participating in its catalytic action on the vapor-phase catalytic oxidation reaction, and is characterized in that, in the step of preparing it by extrusion-molding previously prepared catalyst particles containing at least molybdenum, bismuth and iron, a ceramic material is used for at least a part of the catalyst flow path in this extrusion molding step. The extrusion-molded catalyst thus obtained exhibits higher catalytic activity and higher selectivity for the unsaturated aldehyde and unsaturated carboxylic acid being desired products, as compared with the case where a conventional catalyst flow path made of metal is used. That is, according to the process of the preparation of an extrusion-molded catalyst in accordance with the present invention which employs a simple means comprising using a ceramic material for at least a part of the catalyst flow path in the extrusion molding step, the resulting extrusion-molded catalyst can achieve a further improvement in catalytic activity and in selectivity for the unsaturated aldehyde and unsaturated carboxylic acid being desired products, as compared with catalysts prepared by using a conventional catalyst flow path made of metal. Utilizing the above-described advantages, the extrusion-molded catalyst of the present invention may be applied to a process for the synthesis of an unsaturated aldehyde and an unsaturated carboxylic acid by a vapor-phase catalytic oxidation reaction using propylene, isobutylene, tert-butyl alcohol or methyl tert-butyl ether as a raw material and molecular oxygen as an oxygen source. Thus, by using the extrusion-molded catalyst of the present invention as a catalyst for this vapor-phase catalytic oxidation reaction, the unsaturated aldehyde and the unsaturated carboxylic acid can be produced in higher yield.

### Exploitability in Industry

The catalysts for the synthesis of an unsaturated aldehyde and an unsaturated carboxylic acid in accordance with the present invention have high catalytic activity and high selectivity for the unsaturated aldehyde and unsaturated carboxylic acid being synthesized. The use of these catalysts makes it possible to produce unsaturated aldehydes and unsaturated carboxylic acids in high yield.

1. A catalyst for the synthesis of an unsaturated aldehyde and an unsaturated carboxylic acid characterized in that said catalyst is an extrusion-molded catalyst comprising at least molybdenum, bismuth and iron as metallic elements participating in its catalytic action on the vapor-phase catalytic oxidation reaction in which it is usable to catalyzes the vapor-phase catalytic oxidation reaction for the synthesis of the unsaturated aldehyde and the unsaturated carboxylic acid by using propylene, isobutylene, tert-butyl alcohol or methyl tert-butyl ether as a raw material and using molecular oxygen as an oxygen source, and
said extrusion-molded catalyst being extruded in the step where, when previously prepared catalyst particles containing at least molybdenum, bismuth and iron are subjected to extrusion molding, a ceramic material is used for at least a part of the catalyst flow path in said step of extrusion-molding a kneaded material containing said catalyst particles.

2. A catalyst as claimed in claim 1 wherein said kneaded material is obtained by adding a β-1,3-glucan and a liquid to said catalyst particles and kneading the resulting mixture.

3. A catalyst as claimed in claim 1 wherein said kneaded material is obtained by adding a β-1,3-glucan, a cellulose derivative and a liquid to said catalyst particles and kneading the resulting mixture.

4. A catalyst as claimed in any one of claims 1 to 3 wherein the ceramic material used for at least a part of the catalyst flow path in the extrusion molding step is a ceramic material selected from the group consisting of zirconia, alumina, silica, titania and mixtures of two or more of these materials.

5. A process for preparing an extrusion-molded catalyst for the synthesis of an unsaturated aldehyde and an unsaturated carboxylic acid characterized in that
said catalyst is an extrusion-molded catalyst comprising at least molybdenum, bismuth and iron as metallic elements participating in its catalytic action on the vapor-phase catalytic oxidation reaction in which it is usable to catalyzes the vapor-phase catalytic oxidation reaction for the synthesis of the unsaturated aldehyde and the unsaturated carboxylic acid by using propylene, isobutylene, tert-butyl alcohol or methyl tert-butyl ether as a raw material and using molecular oxygen as an oxygen source; and
said process comprising the steps of kneading previously prepared catalyst particles with a liquid medium, and extruding the resulting kneaded material through a predetermined catalyst flow path to form it into a desired shape,
wherein a ceramic material is used for at least a part of the catalyst flow path for said extrusion molding step.

6. A process for preparing a catalyst for the synthesis of an unsaturated aldehyde and an unsaturated carboxylic acid as claimed in claim 5 wherein in the step of kneading said catalyst particles with a liquid medium, said catalyst particles are kneaded with a liquid medium and a β-1,3-glucan.

7. A process for preparing a catalyst for the synthesis of an unsaturated aldehyde and an unsaturated carboxylic acid as claimed in claim 6 wherein in the step of kneading said catalyst particles with a liquid medium, said catalyst particles are kneaded with a liquid medium, a β-1,3-glucan and a cellulose derivative.

8. A process for the synthesis of an unsaturated aldehyde and an unsaturated carboxylic acid characterized in that in said process, the unsaturated aldehyde and the unsaturated carboxylic acid is synthesized by a vapor-phase catalytic oxidation using propylene, isobutylene, tert-butyl alcohol or methyl tert-butyl ether as a raw material and using molecular oxygen as an oxygen source, and
the catalyst of any one of claims 1 to 3 is used as a catalyst for said vapor-phase catalytic oxidation reaction.

9. A process for the synthesis of an unsaturated aldehyde and an unsaturated carboxylic acid characterized in that in said process, the unsaturated aldehyde and the unsaturated carboxylic acid is synthesized by a vapor-phase catalytic oxidation using propylene, isobutylene, tert-butyl alcohol or methyl tert-butyl ether as a raw material and using molecular oxygen as an oxygen source, and
the catalyst of claim 4 is used as a catalyst for said vapor-phase catalytic oxidation reaction.

10. A catalyst for the synthesis of an unsaturated aldehyde and an unsaturated carboxylic acid characterized in that said catalyst is an extrusion-molded catalyst comprising at least molybdenum, bismuth and iron as metallic elements participating in its catalytic action on the vapor-phase catalytic oxidation reaction in which it is usable to catalyzes the vapor-phase catalytic oxidation reaction for the synthesis of the unsaturated aldehyde and the unsaturated carboxylic acid by using propylene, isobutylene, tert-butyl alcohol or methyl tert-butyl ether as a raw material and using molecular oxygen as an oxygen source, and
said extrusion-molded catalyst being extruded in the step where, when previously prepared catalyst particles containing at least molybdenum, bismuth and iron are subjected to extrusion molding, a kneaded material is prepared by adding a β-1,3-glucan and a liquid to said catalyst particles and kneading the resulting mixture, and then subjected to the extrusion molding.

11. A catalyst for the synthesis of an unsaturated aldehyde and an unsaturated carboxylic acid characterized in that said catalyst is an extrusion-molded catalyst comprising at least molybdenum, bismuth and iron as metallic elements participating in its catalytic action on the vapor-phase catalytic oxidation reaction in which it is usable to catalyzes the vapor-phase catalytic oxidation reaction for the synthesis of the unsaturated aldehyde and the unsaturated carboxylic acid by using propylene, isobutylene, tert-butyl alcohol or methyl tert-butyl ether as a raw material and using molecular oxygen as an oxygen source, and
said extrusion-molded catalyst being extruded in the step where, when previously prepared catalyst particles containing at least molybdenum, bismuth and iron are subjected to extrusion molding, a kneaded material is prepared by adding a β-1,3-glucan, a cellulose derivative and a liquid to said catalyst particles and kneading the resulting mixture, and then subjected to the extrusion molding.

12. A catalyst as claimed in claim 10 wherein said liquid is water.

13. A catalyst as claimed in claim 11 wherein said liquid is water.

14. A catalyst as claimed in any one of claims 10 to 13 wherein said β-1,3-glucan is curdlan.

15. A catalyst as claimed in any one of claims 11 to 13 wherein said cellulose derivative comprises one or more members selected from the group consisting of methylcellulose, carboxymethylcellulose, hydroxypropyl methylcellulose and hydroxyethyl methylcellulose.

16. A catalyst as claimed in claim 14 wherein said cellulose derivative comprises one or more members selected from the group consisting of methylcellulose, carboxymethylcellulose, hydroxypropyl methylcellulose and hydroxyethyl methylcellulose.

17. A process for preparing an extrusion-molded catalyst for the synthesis of an unsaturated aldehyde and an unsaturated carboxylic acid characterized in that
said catalyst is an extrusion-molded catalyst comprising at least molybdenum, bismuth and iron as metallic elements participating in its catalytic action on the vapor-phase catalytic oxidation reaction in which it is usable to catalyzes the vapor-phase catalytic oxidation reaction for the synthesis of the unsaturated aldehyde and the unsaturated carboxylic acid by using propylene, isobutylene, tert-butyl alcohol or methyl tert-butyl ether as a raw material and using molecular oxygen as an oxygen source; and
said process comprising the steps of adding a β-1,3-glucan and a liquid to previously prepared catalyst particles containing molybdenum, bismuth and iron, and kneading the resulting mixture; and extruding the resulting kneaded material into a desired shape.

18. A process for preparing an extrusion-molded catalyst for the synthesis of an unsaturated aldehyde and an unsaturated carboxylic acid characterized in that
said catalyst is an extrusion-molded catalyst comprising at least molybdenum, bismuth and iron as metallic elements participating in its catalytic action on the vapor-phase catalytic oxidation reaction in which it is usable to catalyzes the vapor-phase catalytic oxidation reaction for the synthesis of the unsaturated aldehyde and the unsaturated carboxylic acid by using propylene, isobutylene, tert-butyl alcohol or methyl tert-butyl ether as a raw material and using molecular oxygen as an oxygen source; and
said process comprising the steps of adding a β-1,3-glucan, a cellulose derivative and a liquid to previously prepared catalyst particles containing molybdenum, bismuth and iron, and kneading the resulting mixture; and extrusion-molding the resulting kneaded material into a desired shape.

19. A process for the synthesis of an unsaturated aldehyde and an unsaturated carboxylic acid characterized in that in said process, the unsaturated aldehyde and the unsaturated carboxylic acid is synthesized by a vapor-phase catalytic oxidation using propylene, isobutylene, tert-butyl alcohol or methyl tert-butyl ether as a raw material and using molecular oxygen as an oxygen source, and
the catalyst of any one of claims 10 to 13 is used as a catalyst for said vapor-phase catalytic oxidation reaction.

20. A process for the synthesis of an unsaturated aldehyde and an unsaturated carboxylic acid characterized in that in said process, the unsaturated aldehyde and the unsaturated carboxylic acid is synthesized by a vapor-phase catalytic oxidation using propylene, isobutylene, tert-butyl alcohol or methyl tert-butyl ether as a raw material and using molecular oxygen as an oxygen source, and
the catalyst of claim 14 is used as a catalyst for said vapor-phase catalytic oxidation reaction.

21. A process for the synthesis of an unsaturated aldehyde and an unsaturated carboxylic acid characterized in that in said process, the unsaturated aldehyde and the unsaturated carboxylic acid is synthesized by a vapor-phase catalytic oxidation using propylene, isobutylene, tert-butyl alcohol or methyl tert-butyl ether as a raw material and using molecular oxygen as an oxygen source, and
the catalyst of claim 15 is used as a catalyst for said vapor-phase catalytic oxidation reaction.

22. A process for the synthesis of an unsaturated aldehyde and an unsaturated carboxylic acid characterized in that in said process, the unsaturated aldehyde and the unsaturated carboxylic acid is synthesized by a vapor-phase catalytic oxidation using propylene, isobutylene, tert-butyl alcohol or methyl tert-butyl ether as a raw material and using molecular oxygen as an oxygen source, and
the catalyst of claim 16 is used as a catalyst for said vapor-phase catalytic oxidation reaction.
